(19) United States
(12) Patent Application Publication
(10) Pub. No.: US 2022/0023812 A1
(43) Pub. Date: Jan. 27, 2022

(54) Bicyclic Surfactants

(71) Applicant: ExxonMobil Chemical Patents Inc., Baytown, TX (US)

(72) Inventors: Mika L. Shiramizu, Houston, TX (US); Alan A. Galuska, Ellijay, GA (US); Jennifer A. Carvajal Diaz, Spring, TX (US)

(21) Appl. No.: 17/311,725

(22) PCT Filed: Nov. 20, 2019
(86) PCT No.: PCT/US2019/062324

§ 371 Date: Jun. 8, 2021

Related U.S. Application Data
(60) Provisional application No. 62/781,637, filed on Dec. 19, 2018.

(30) Foreign Application Priority Data
Mar. 4, 2019 (EP) 19160492.5

(51) Int. Cl. B01F 17/00; C07C 309/37; C07C 309/31

(52) U.S. Cl. CPC B01F 17/0057; C07C 309/37; C07C 2602/10; C07C 2601/16; C07C 309/31

(57) ABSTRACT

Fused and unfused bicyclic rings are a convenient scaffold for synthesizing various compounds. Amphiphilic compounds constructed upon a fused or unfused bicyclic ring scaffold may exhibit surfactant properties differing from those of more conventional classes of surfactants. Such amphiphilic compounds may have a structure of formula (I), wherein A is an aromatic ring, a heteroaromatic ring, or a cycloaliphatic ring, each ring being defined by 5 to 10 ring atoms, B is an aromatic ring or a heteroaromatic ring, each ring being defined by 5 to 6 ring atoms, wherein at least one G is a hydrophobic moiety and at least one G is a hydrophilic moiety, and z is 0 or a positive integer ranging up to the number of ring atoms in each of A and B; and wherein the hydrophobic moiety comprises branched or unbranched C₆ to C₃₀ alkyl or alkenyl group, and the hydrophilic moiety comprises a polar functional group selected from the group consisting of quaternary ammonium, sulfonate, sulfate, carboxylate, phosphate, and ethoxylated.

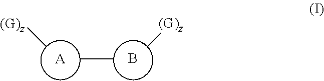

### CROSS-REFERENCE TO RELATED APPLICATIONS

This application claims the benefit of Provisional Application No. 62/781,637, filed Dec. 19, 2018 and European Application No. 19160492.5, filed Mar. 4, 2019, the disclosures of which are incorporated herein by reference.

### FIELD

The present disclosure relates to surfactants formed upon a scaffold bearing two or more carbocyclic and/or heterocyclic rings, which may be fused or unfused.

### BACKGROUND

Surfactants are compounds that tend to lower the surface tension at an interface between two components. As such, surfactants may be used in a wide range of applications, which may include, for example, promoting solubility of an otherwise sparingly soluble solid, lowering viscosity of a fluid phase, and promoting foaming of a fluid. Surfactants may be found in a wide range of consumer and industrial products including, for example, soaps, detergents, cosmetics, pharmaceuticals, and dispersants.

Surfactants feature both hydrophobic and hydrophilic regions within their molecular structure. Hydrophobic regions are generally non-ionic and may include saturated or unsaturated hydrocarbyl groups, such as alkyl, alkenyl, or aryl groups. Hydrophilic regions, in contrast, may be ionic, non-ionic, or zwitterionic and encompass a range of polar functional groups or moieties. Ionic functional groups that may be present in the hydrophilic regions of various surfactants include, for example, sulfonates, sulfates, carboxylates, phosphates, quaternary ammonium groups, and the like. Non-ionic hydrophilic regions may include functional groups or moieties bearing one or more heteroatoms that are capable of receiving hydrogen bonds, such as polyethers (e.g., ethoxylates). Zwitterionic hydrophilic regions may include moieties such as betaines, sultaines, and related phospholipid compounds.

Surfactants finding extensive commercial use feature a relatively limited range of structure types. Common classes of commercial surfactants include, for example, alkylbenzene sulfonates, lignin sulfonates, long chain fatty alcohol sulfates, long chain fatty acid carboxylates, long chain fatty alcohol ethoxylates, long chain quaternary ammonium compounds, and alkylphenol ethoxylates. The various classes of surfactants may exhibit a range of surfactant properties, and there may be further property variation within the members or homologues within each class. Accordingly, a surfactant for a given application may be chosen based upon various application-specific requirements. There remains a need, however, for development of additional types of surfactants having different structural features to facilitate presently unmet or presently unknown application-specific requirements within various industries.

Relevant publications include CN 105800909B; CN 106212456B; CN 108707262A; WO 2015/106174; and WO 2018/157269.

### SUMMARY

In any embodiment, the present disclosure provides surfactant compositions based upon an unfused bicyclic ring molecular scaffold. The surfactant compositions comprise one or more amphiphilic compounds having a structure of

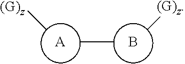

wherein A is an aromatic ring, a heteroaromatic ring, or a cycloaliphatic ring, each ring being defined by 5 to 10 ring atoms, B is an aromatic ring or a heteroaromatic ring, each ring being defined by 5 to 6 ring atoms, wherein at least one G is a hydrophobic moiety and at least one G is a hydrophilic moiety, and z is 0 or a positive integer ranging up to the number of ring atoms in each of A and B; and wherein the hydrophobic moiety comprises branched or unbranched C₆ to C₃₀ alkyl or alkenyl group, and the hydrophilic moiety comprises a polar functional group selected from the group consisting of quaternary ammonium, sulfonate, sulfate, carboxylate, phosphate, and ethoxylated alkyls (“ethoxylate”), preferably sulfonate. In any embodiment, such branched or unbranched C₆ to C₃₀ alkyl or alkenyl group are preferably internal vinylidenes before alkylating to the one or more rings, “internal” meaning the unsaturation is not located at the terminal carbon atoms of the alkylene, most preferably C₆ to C₂₀, or C₃₀ internal vinylidenes.

### DETAILED DESCRIPTION

The present disclosure generally relates to surfactants and, more specifically, to surfactants based upon a bicyclic molecular scaffold featuring fused or unfused rings.

As discussed above, the majority of surfactants in common commercial use are based upon a relatively limited number of chemical structural classes, including alkylbenzene sulfonates, lignin sulfonates, long chain fatty alcohol sulfates, long chain fatty acid carboxylates, long chain fatty alcohol ethoxylates, long chain quaternary ammonium compounds, and alkylphenol ethoxylates. The various structural classes, as well as specific members or homologues within each structural class, may exhibit a range of surfactant properties, which may be chosen for suitability or compatibility with a given application. Some existing and emerging applications may have application-specific needs that are not adequately met by presently available surfactants. For example, certain conventional surfactants may not exhibit a desired toxicity or biodegradation profile, and/or provide sufficient surface modification effects in some instances, such as under elevated thermal conditions.

The present disclosure describes various classes of amphiphilic compounds that are constructed upon a molecular scaffold comprising at least two fused or unfused rings (i.e., bicyclic molecular scaffolds). Suitable bicyclic molecular scaffolds may include, for example, fused aromatic compounds containing two or more fused rings, such as naphthalenes, dihydronaphthalenes, tetrahydronaphthalenes, fluorenes, phenanthrenes, dihydrophenanthrenes, anthracenes, and dihydroanthracene, and unfused bicyclic compounds, such as biaryl-type compounds (including heteroaryl variants such as bis-furans and bis-thiophenes) and aryl compounds bearing at least one unfused cycloaliphatic ring (e.g., cyclopentylbenzene, cyclopenten-1-ylbenzene, cyclohexylbenzene, cyclohexen-1-ylbenzene, and the like). Particular examples are provided herein below.

The amphiphilic compounds disclosed herein may be particularly advantageous because of the synthetic versatility of the aromatic and heteroaromatic rings defining their molecular scaffolds. Aromatic and heteroaromatic rings may undergo relatively controlled synthetic modifications to introduce thereto the hydrophobic and hydrophilic moieties needed for conveying surfactant properties. A multitude of reaction conditions are available for functionalizing aromatic and heteroaromatic rings with various functional groups in a desired location (ring position). Because of the rich synthetic diversity of aromatic and heteroaromatic rings, numerous combinations of hydrophobic and hydrophilic moieties are possible in the amphiphilic compounds disclosed herein. Moreover, since aromatic and heteroaromatic rings have a limited number of valence positions (bonding locations) disposed in relatively fixed positions with respect to one another, various geometric arrangements of the hydrophobic and hydrophilic moieties may be present in the amphiphilic compounds disclosed herein. The fixed geometric arrangements may promote surfactant properties in the amphiphilic compounds of the present disclosure that are not presently exhibited by surfactants in common commercial use. Variation of the geometric arrangement of the hydrophobic and hydrophilic moieties may promote further tailoring of the surfactant properties.

As a particular example, at least some of the amphiphilic compounds disclosed herein may exhibit very high tolerance toward hard water, as measured by calcium tolerance measurements (resistance to precipitation or hazing in the presence of calcium). Many conventional surfactants, in contrast, display high propensity toward precipitation or hazing in the presence of alkaline earth metal cations, such as calcium. The high calcium tolerance offered by the amphiphilic compounds disclosed herein may promote surfactant compatibility in applications where surfactants are inefficiently used at present (due to precipitation) or are not feasible for use at all. Some of the amphiphilic compounds disclosed herein also exhibit relatively high C₂₀ values compared to conventional surfactants, which may provide further to advantages.

In addition, some bicyclic molecular scaffolds featuring fused or unfused rings may be derived from biological sources or at least bear structural similarity to compounds that are derived from biological sources. Bis-furan compounds, for instance, may be derived from biomass or other biological sources. The relationship of certain molecular scaffolds of the present disclosure to biologically derived compounds may afford an improved environmental profile relative to other types of surfactant compounds. In addition, potential biological sourcing of the molecular scaffold offers the possibility for low-cost syntheses of certain amphiphilic compounds disclosed herein.

Unless otherwise indicated, room temperature is 25° C.

As used in the present disclosure and claims, the singular forms “a,” “an,” and “the” include plural forms unless the context clearly dictates otherwise.

The term “and/or” as used in a phrase such as “A and/or B” herein is intended to include “A and B,” “A or B,” “A”, and “B.”

For the purposes of the present disclosure, the new numbering scheme for groups of the Periodic Table is used. In said numbering scheme, the groups (columns) are numbered sequentially from left to right from 1 through 18, excluding the f-block elements (lanthanides and actinides).

The term “hydrocarbon” refers to a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different numbers of carbon atoms. The term “Cₙ” refers to hydrocarbon(s) or a hydrocarbyl group having n carbon atom(s) per molecule or group, wherein n is a positive integer. Such hydrocarbons or hydrocarbyl groups may be one or more of linear, branched, cyclic, acyclic, saturated, unsaturated, aliphatic, or aromatic.

The terms “saturated” or “saturated hydrocarbon” refer to a hydrocarbon or hydrocarbyl group in which all carbon atoms are bonded to four other atoms or bonded to three other atoms with one unfilled valence position thereon.

The terms “unsaturated” or “unsaturated hydrocarbon” refer to a hydrocarbon or hydrocarbyl group in which one or more carbon atoms are bonded to less than four other atoms, optionally with one unfilled valence position on the one or more carbon atoms.

The terms “hydrocarbyl” and “hydrocarbyl group” are used interchangeably herein. The term “hydrocarbyl group” refers to any C₁-C₁₀₀ hydrocarbon group bearing at least one unfilled valence position when removed from a parent compound. “Hydrocarbyl groups” may be optionally substituted, in which the term “optionally substituted” refers to replacement of at least one hydrogen atom or at least one carbon atom with a heteroatom or heteroatom functional group. Heteroatoms may include, but are not limited to, B, O, N, S, P, F, Cl, Br, I, Si, Pb, Ge, Sn, As, Sb, Se, and Te. Heteroatom functional groups that may be present in substituted hydrocarbyl groups include, but are not limited to, functional groups such as O, S, S═O, S(═O)₂, NO₂, F, Cl, Br, I, NR₂, OR, SeR, TeR, PR₂, AsR₂, SbR₂, SR, BR₂, SiR₃, GeR₃, SnR₃, PbR₃, where R is a hydrocarbyl group or H. Suitable hydrocarbyl groups may include alkyl, alkenyl, alkynyl, aryl, heteroaryl, cycloalkyl, cycloalkenyl, heterocyclyl, and the like, any of which may be optionally substituted.

The term “alkyl” refers to a hydrocarbyl group having no unsaturated carbon-carbon bonds, and which may be optionally substituted. The term “alkylene” refers to an alkyl group having at least two open valence positions.

The term “alkenyl” refers to a hydrocarbyl group having a carbon-carbon double bond, and which may be optionally substituted. The terms “alkene” and “olefin” may be used synonymously herein. Similarly, the terms “alkenic” and “olefinic” may be used synonymously herein. Unless otherwise noted, all possible geometric isomers are encompassed by these terms.

The term “cycloaliphatic ring” refers to a saturated or unsaturated hydrocarbyl ring. Examples of cycloaliphatic rings include, for example, cycloalkyl rings and cycloalkenyl rings.

The terms “aromatic” and “aromatic hydrocarbon” refer to a hydrocarbon or hydrocarbyl group having a cyclic arrangement of conjugated pi-electrons that satisfy the Hückel rule. The term “aryl” is equivalent to the term “aromatic” as defined herein. The term “aryl” refers to both aromatic compounds and heteroaromatic compounds, either of which may be optionally substituted. Both mononuclear and polynuclear aromatic compounds are encompassed by these terms.

The terms “heteroaromatic” and “heteroaryl” refer to an aromatic compound having at least one heteroatom within the ring defined therein. Both mononuclear and polynuclear heteroaromatic compounds are encompassed by these terms. Heteroaryl groups may include, but are not limited to, pyridine, quinoline, isoquinoline, pyrimidine, quinazoline, acridine, pyrazine, quinoxaline, imidazole, benzimidazole, pyrazole, benzopyrazole, oxazole, benzoxazole, isoxazole, benzisoxazole, imidazoline, thiophene, benzothiophene, furan and benzofuran.

Examples of saturated hydrocarbyl groups include, but are not limited to, methyl, ethyl, n-propyl, isopropyl, n-butyl, isobutyl, sec-butyl, tert-butyl, pentyl, isopentyl, hexyl, octyl, cyclopropyl, cyclobutyl, cyclopentyl, cyclohexyl, cyclooctyl, and the like, including their substituted analogues. Examples of unsaturated hydrocarbyl groups include, but are not limited to, ethenyl, propenyl, allyl, butadienyl, cyclopropenyl, cyclobutenyl, cyclopentenyl, cyclohexenyl, cyclooctenyl and the like, including their substituted analogues.

Examples of aromatic hydrocarbyl groups include, but are not limited to, phenyl, tolyl, xylyl, naphthyl, and the like, including all possible isomeric forms thereof. Polynuclear aromatic hydrocarbyl groups may include, but are not limited to, naphthalene, anthracene, indane, indene, and tetralin.

The terms “linear” and “linear hydrocarbon” refer to a hydrocarbon or hydrocarbyl group having a continuous carbon chain without side chain branching, in which the continuous carbon chain may be optionally substituted with heteroatoms or heteroatom groups.

The terms “branch,” “branched” and “branched hydrocarbon” refer to a hydrocarbon or hydrocarbyl group having a linear main carbon chain in which a hydrocarbyl side chain extends from the linear main carbon chain. Optional heteroatom substitution may be present in the linear main carbon chain or in the hydrocarbyl side chain.

The term “amphiphilic compound” refers to a compound having both a hydrophobic moiety and a hydrophilic moiety within its molecular structure.

The term “ring atoms” refers to a plurality of atoms joined together in a loop to form a closed ring structure. Additional functional groups, rings, and/or chains may extend from one or more of the ring atoms, with atoms in the additional functional groups, rings, and/or chains not being included in the count of ring atoms.

As used herein, the term “ethoxylate” refers to the moiety —(CH₂CH₂O)ₓ—, wherein x is an integer ranging from 3 to 20.

Surfactant compositions of the present disclosure may comprise one or more amphiphilic compounds having a structure corresponding to Formula 1.

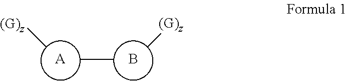

Referring to Formula 1, A is an aromatic ring, a heteroaromatic ring, or a cycloaliphatic ring, each ring being defined by 5 to 10 ring atoms, B is an aromatic ring or a heteroaromatic ring, each ring being defined by 5 to 6 ring atoms, wherein at least one G is a hydrophobic moiety and at least one G is a hydrophilic moiety, and z is 0 or a positive integer ranging up to the number of ring atoms in each of A and B; and wherein the hydrophobic moiety comprises to branched or unbranched C₆ to C₃₀ alkyl or alkenyl group, and the hydrophilic moiety comprises a polar functional group selected from the group consisting of quaternary ammonium, sulfonate, sulfate, carboxylate, phosphate, and ethoxylate, preferably sulfonate. In any embodiment, such branched or unbranched C₆ to C₃₀ alkyl or alkenyl groups are preferably internal vinylidenes before reacting, meaning the unsaturation is not located at the terminal carbon atoms of the alkylene, most preferably C₆ to C₂₀, or C₃₀ internal vinylidenes.

Referring still to Formula 1, a hydrophobic moiety extends from at least one of ring A or ring B, and a hydrophilic moiety extends from at least one of ring A or ring B. The hydrophobic moiety comprises a branched or unbranched, saturated or unsaturated hydrocarbyl group, and at least one hydrophobic moiety extends from at least one of ring A or ring B. The hydrophilic moiety comprises a polar functional group selected from the group consisting of quaternary ammonium, sulfonate, sulfate, carboxylate, phosphate, and ethoxylate, and at least one hydrophilic moiety extends from at least one of ring A or ring B. Some surfactant compositions of the present disclosure having Formula 1 feature only sulfonate groups as the hydrophilic moiety upon ring A and/or ring B. Bonding locations for the hydrophobic moiety and the hydrophilic moiety are not particularly limited and may occur at any open valence position upon ring A or ring B, as discussed further below.

Alternately, ring A and ring B may be fused together, such that at least some of the ring atoms are shared in common between the two rings. Formula 2 shows a structure of fused-ring amphiphilic compounds that may be present in the surfactant compositions of the present disclosure. Most typically, two or three ring atoms are shared in common between the two rings.

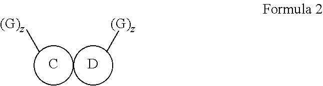

Referring still to Formula 2, a hydrophobic moiety extends from at least one of ring C or ring D, and a hydrophilic moiety extends from at least one of ring C or ring D. Ring C is defined by 5 to 6 ring atoms. Ring D is defined by 5 to 6 ring atoms. The hydrophobic moieties and the hydrophilic moieties that may be present in Formula 2 are the same as those that may be present in Formula 1, as defined further above. Some surfactant compositions of the present to disclosure having Formula 2 feature only sulfonate groups as the hydrophilic moiety upon ring C and/or ring D. Likewise, variable z is defined as above for Formula 1, and bonding locations for the hydrophobic moiety and the hydrophilic moiety are not particularly limited and may occur at any open valence position upon ring C and/or ring D.

Particular combinations of rings encompassing ring A and ring B in Formula 1 are shown in Formulas 3-7 below, wherein at least one hydrophobic moiety extends from at least one of ring A or ring B and at least one hydrophilic moiety extends from at least one of ring A or ring B. The hydrophobic moiety comprises a branched or unbranched, saturated or unsaturated hydrocarbyl group. The hydrophilic moiety comprises a polar functional group selected from the group consisting of quaternary ammonium, sulfonate, sulfate, carboxylate, phosphate, and ethoxylate, preferably sulfonate. Sulfonate may be the polar functional group(s) in some instances. Again, suitable bonding locations for the hydrophobic moiety and the hydrophilic moiety are not particularly limited and may occur at any open valence position of either ring, as discussed further below.

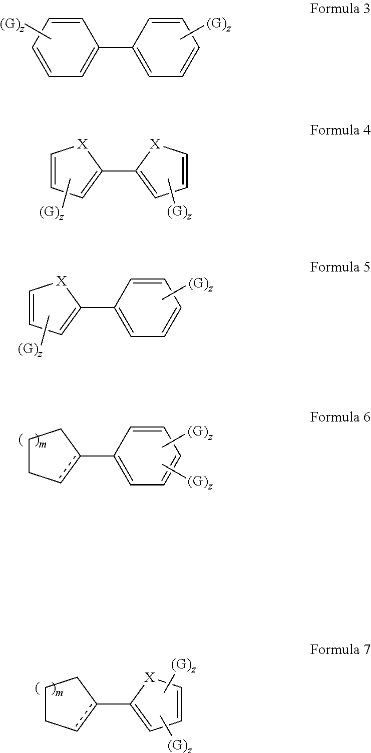

Formulas 6 and 7 show structures in which cycloaliphatic rings of varying sizes and states of unsaturation may be present in the bicyclic molecular scaffold. In particular, variable m may be an integer ranging from 1 to 6, such that the cycloaliphatic ring contains between 5 and 10 carbon atoms. The cycloaliphatic ring may also contain optional unsaturation as well, as indicated by the dashed double bond in Formulas 6 and 7. When present, the double bond may include one carbon atom that is sigma bonded to the phenyl ring (Formula 6) or heteroaromatic ring (Formula 7). The unsaturated variants of Formulas 6 and 7 may be hydrogenated in some instances to form the corresponding saturated variants in some instances. Other double bond regioisomeric variants of Formulas 6 and 7 also lie within the scope of the present disclosure.

In Formulas 4 and 7, X may be 0 or S, such that the heteroaromatic ring is a furan or a thiophene.

In Formulas 6 and 7, the at least one hydrophobic moiety and the at least one hydrophilic moiety both extend from the phenyl ring (Formula 6) or the heteroaromatic ring (Formula 7), thereby leaving the cycloaliphatic ring unsubstituted. In Formulas 3-5, in contrast, the at least one hydrophobic moiety and the at least one hydrophilic moiety may extend from either phenyl ring and/or heteroaromatic ring. Moreover, the at least one hydrophobic moiety and the at least one hydrophilic moiety may be present upon the same ring or upon different rings. More than one hydrophobic moiety and/or more than one hydrophilic moiety may be present upon either ring in some instances. Again, bonding locations for the at least one hydrophobic moiety and the at least one hydrophilic moiety are not particularly limited and may occur at any open valence position upon either ring, as discussed further below.

In more specific embodiments, the one or more amphiphilic compounds in the surfactant compositions of the present disclosure may have a structure defined by Formula 8 below.

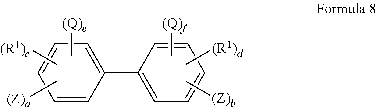

Referring to Formula 8, R¹ is a hydrophobic moiety that is a branched or unbranched C₆ to C₃₀ or C₈ to C₂₄ alkyl or alkenyl group, where each occurrence of R¹ is the same or different. Particular examples of alkyl or alkenyl groups that may be present as R¹ include, for example, n-hexyl, n-octyl, n-decyl, n-dodecyl, n-eicosyl, 2-methylhexyl, 2-methyloctyl, hexenyl, octenyl, decenyl, and the like.

One or more hydrophobic moieties defined by R¹ may be present in Formula 8. In to particular embodiments, a and b are integers greater than or equal to zero, with the proviso that at least one of a or b is not zero.

Z is a hydrophilic moiety that is selected from the group consisting of quaternary ammonium, sulfonate, sulfate, carboxylate, phosphate, and ethoxylate, where each occurrence of Z is the same or different. In some instances, each occurrence of Z may be sulfonate.

One or more hydrophilic moieties defined by Z may be present in Formula 8. In particular embodiments, c and d are integers greater than or equal to zero, with the proviso that at least one of c or d is not zero.

Q is a functional group that may be optionally present in addition to the at least one hydrophobic moiety and the at least one hydrophilic moiety. Selections for Q include, for example, a C₁ to C₄ alkyl group, a C₁ to C₄ alkoxy group, or a halide. Zero or one functional groups defined by Q may be present upon each phenyl ring in Formula 8. That is, variables e and f are independently selected from 0 or 1. Each occurrence of Q is the same or different. When no functional group Q is present, a C—H bond is present in its place.

Multiple occurrences of R¹ and Z may be present upon each phenyl ring in Formula 8, up to the maximum available valency. That is, the sum a+c+e is 5 or less and the sum b+d+f is also 5 or less.

Combinations of R¹ and Z that may be present include those shown in Formulas 9-21 below, in which one R¹ and one Z may be present, or one R¹ and two Z may be present, or two R¹ and one Z may be present, or two R¹ and two Z may be present, or three R¹ and one Z may be present, or three R¹ and two Z may be present, or four R¹ and one Z may be present, or four R¹ and two Z may be present. The bonding locations for particular combinations of R¹ and Z may be positioned upon either phenyl ring and in any possible substitution pattern. Non-specific substitution patterns are shown in Formulas 9-21 below, but it is to be appreciated that even more specific examples may feature R¹, Z and Q at any open valence position upon either phenyl ring.

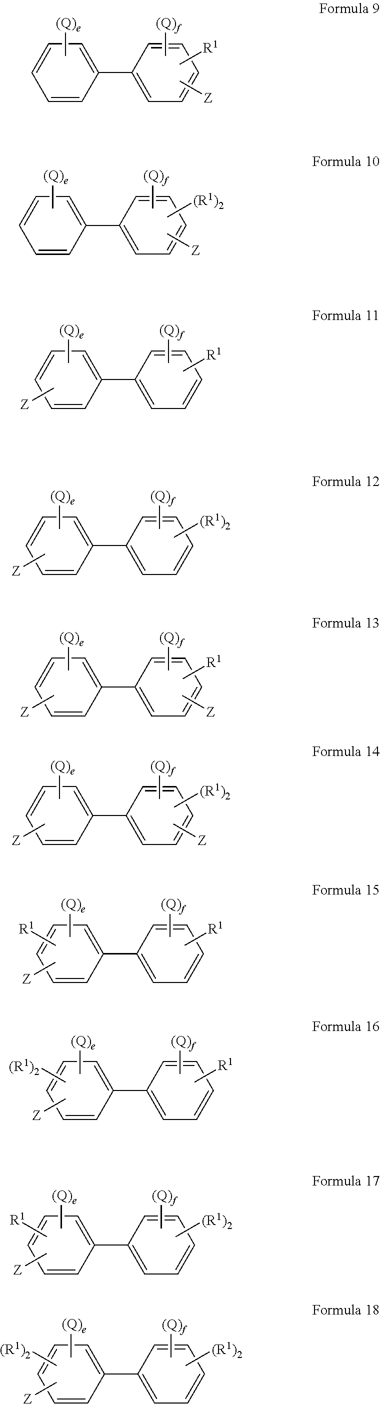

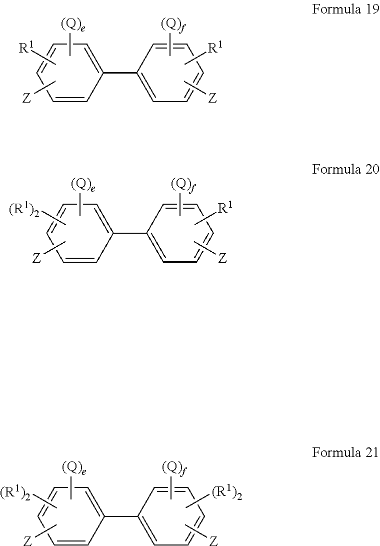

Particular embodiments of Formulas 9-21 include those in which at least one of variables e or f is zero. In any embodiment, variables e and f are both zero.

In other more specific embodiments, the one or more amphiphilic compounds in the surfactant compositions of the present disclosure may have a structure defined by Formula 22 below.

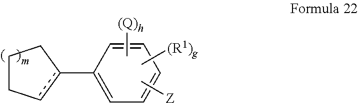

Referring to Formula 22, R¹, Z and Q are defined as above for Formulas 8-21. Particular examples of alkyl or alkenyl groups that may be present as R¹ include, for example, n-hexyl, n-octyl, n-decyl, n-dodecyl, eicosyl, 2-methylhexyl, 2-methyloctyl, hexenyl, octenyl, decenyl, and the like. Z may be sulfonate in particular embodiments.

One or more hydrophobic moieties defined by R¹ may be present. In particular embodiments, one or two hydrophobic moieties are present such that variable g is 1 or 2 in Formula 22. Each occurrence of R¹ is the same or different.

One occurrence of the hydrophilic moiety defined by Z is present in Formula 22.

Zero or one occurrences of functional group Q are present in Formula 22. Thus, variable h is 0 or 1. When no functional group Q is present, a C—H bond is present in its place.

Variable m in Formula 22 is an integer ranging from 1 to 6, thereby forming a optionally unsaturated 5- to 10-membered cycloaliphatic ring. The optionally unsaturated 5- to 10-membered cycloaliphatic ring is selected from the group consisting of cyclopentyl, to cyclohexyl, cycloheptyl, cyclooctyl, cyclononyl, cyclodecyl, cyclopenten-1-yl, cyclohexen-1-yl, cyclohepten-1-yl, cycloocten-1-yl, cyclononen-1-yl, and cyclodecen-1-yl. Other double bond regioisomers also lie within the scope of the present disclosure. In particular embodiments, the cycloaliphatic ring may be saturated, such that the one or more amphiphilic compounds have a structure selected from among Formulas 22a and 22b below.

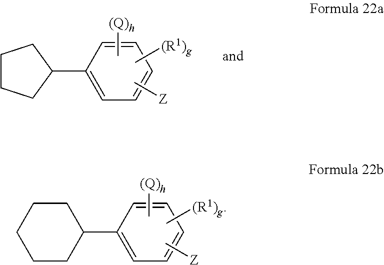

In still other more specific embodiments, the one or more amphiphilic compounds in the surfactant compositions of the present disclosure may have a structure defined by Formula 23 below.

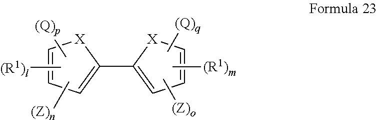

Referring to Formula 23, R′, Z and Q are defined as above for Formulas 8-22, 22a and 22b. Particular examples of alkyl or alkenyl groups that may be present as R′ include, for example, n-hexyl, n-octyl, n-decyl, n-dodecyl, eicosyl, 2-methylhexyl, 2-methyloctyl, hexenyl, octenyl, decenyl, and the like.

One or more hydrophobic moieties defined by R¹ may be present. In particular embodiments, l and m are integers greater than or equal to zero, with the proviso that at least one of 1 or m is not zero. Each occurrence of R¹ is the same or different.

One or more hydrophilic moieties defined by Z may be present in Formula 23. In particular embodiments, n and o are integers greater than or equal to zero, with the proviso that at least one of n or o is not zero. Each occurrence of Z is the same or different. Each occurrence of Z may be sulfonate in particular embodiments.

Zero or one functional groups defined by Q may be present upon each heteroaromatic ring in Formula 23. That is, variables p and q are independently selected from 0 or 1. Each occurrence of Q may be the same or different. When no functional group Q is present, a C—H bond is present in its place.

Referring still to Formula 23, multiple occurrences of R′ and Z may be present upon each heteroaromatic ring, up to the maximum available valency. That is, a sum of n+l+p is 3 or less and a sum of o+m+q is also 3 or less.

In Formula 23, X may be selected from O and S, such that the heteroaromatic ring is a furan or a thiophene ring, respectively. Both heteroaromatic rings may be furan rings (X is O), or both heteroaromatic rings may be thiophene rings (X is S), according to some embodiments. In still other embodiments, amphiphilic compounds having one furan ring and one thiophene ring may be present in the surfactant compositions described herein.

In more particular embodiments, X is O in both heteroaromatic rings, such that the one or more amphiphilic compounds are bis-furan compounds. Combinations of R¹ and Z that may be present include those shown in Formulas 24-36 below, in which one R¹ and one Z may be present, or one R¹ and two Z may be present, or two R¹ and one Z may be present, or two R¹ and two Z may be present, or three R′ and one Z may be present, or three R′ and two Z may be present, or four R¹ and one Z may be present, or four R¹ and two Z may be present. The bonding locations for particular combinations of R¹ and Z may be positioned upon either furan ring and in any possible substitution pattern. Non-specific substitution patterns are shown in Formulas 24-36 below, but it to be appreciated that even more specific examples may feature R¹, Z and Q at any open valence position upon either furan ring. Thiophenes and alternative heterocycles may be substituted similarly with R¹ and Z.

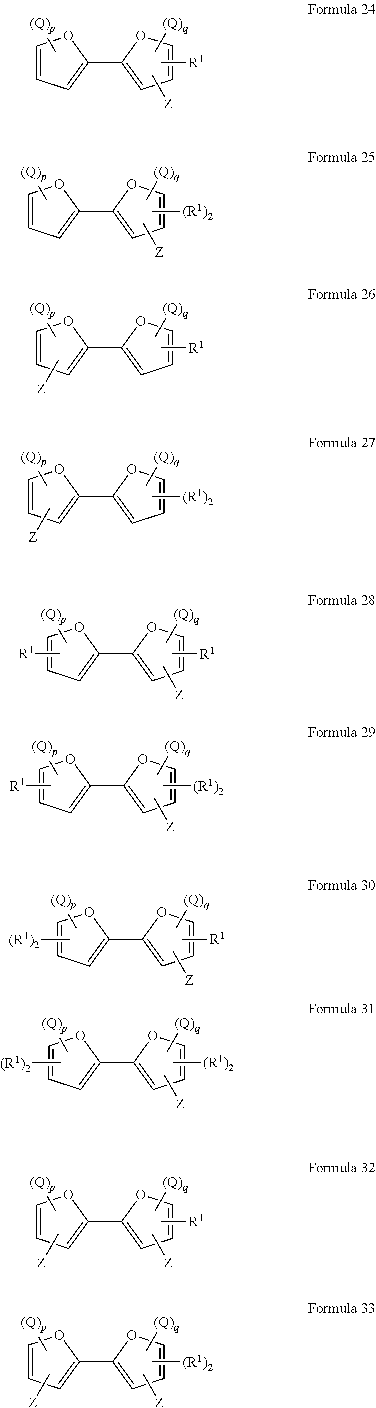

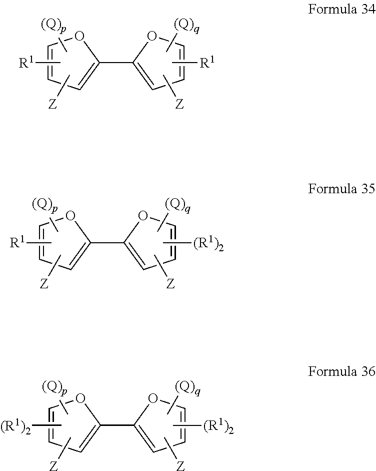

Particular embodiments of Formulas 24-36 include those in which at least one of variables p or q is zero. In some or other embodiments, variables p and q are both zero.

Additional surfactant compositions of the present disclosure may include those in which the one or more amphiphilic compounds feature two aromatic or heteroaromatic rings that are joined together in a fused aromatic or heteroaromatic bicyclic ring scaffold. Fused aromatic and heteroaromatic rings that may be present as bicyclic ring scaffolds according to the present disclosure include, for example, naphthalenes, benzofurans, and benzothiophenes.

In more particular embodiments, the one or more amphiphilic compounds present in the surfactant compositions disclosed herein may be a substituted naphthalene compound to having Formula 37 below.

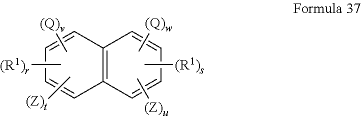

Referring to Formula 37, R¹ is a hydrophobic moiety that is a branched or unbranched C₆ to C₃₀ or C₈ to C₂₄ alkyl or alkenyl group, each occurrence of R¹ being the same or different. Particular examples of alkyl or alkenyl groups that may be present as R¹ include, for example, n-hexyl, n-octyl, n-decyl, n-dodecyl, eicosyl, 2-methylhexyl, 2-methyloctyl, hexenyl, octenyl, decenyl, and the like.

One or more hydrophobic moieties defined by R¹ may be present in Formula 37. In particular embodiments, r and s are integers greater than or equal to zero, with the proviso that at least one of r or s is not zero. Each occurrence of R′ is the same or different.

Z is a hydrophilic moiety that is selected from the group consisting of quaternary ammonium, sulfonate, sulfate, carboxylate, phosphate, and ethoxylate. One or more hydrophilic moieties defined by Z may be present in Formula 37. In particular embodiments, t and u are integers greater than or equal to zero, with the proviso that at least one of t or u is not zero. Each occurrence of Z is the same or different. Each occurrence of Z may be sulfonate in particular embodiments.

Q is a functional group that may be optionally present in addition to the hydrophobic moiety and the hydrophilic moiety in Formula 37. Selections for Q include, for example, a C₁ to C₄ alkyl group, a C₁ to C₄ alkoxy group, and a halide. Zero or one functional groups defined by Q may be present upon each of the fused phenyl rings in Formula 38. That is, variables v and w are independently selected from 0 or 1. Each occurrence of Q is the same or different. When no functional group Q is present, a C—H bond is present in its place.

Referring still to Formula 37, multiple occurrences of R¹ and Z may be present upon each of the fused phenyl rings, up to the maximum available valency. That is, a sum of r+t+v is 4 or less and a sum of s+u+w is also 4 or less.

Specific combinations of R¹ and Z that may be present upon a naphthalene scaffold include those shown in Formulas 38-50 below, in which one R¹ and one Z may be present, or one R′ and two Z may be present, or two R′ and one Z may be present, or two R′ and two Z may be present, or three R′ and one Z may be present, or three R′ and two Z may be present, or four R¹ and one Z may be present, or four R¹ and two Z may be present. The particular combinations of R¹ and Z may be present upon either of the fused phenyl rings of the naphthalene bicyclic scaffold, and the locations are R′ and Z are not limited to any particular substitution pattern. Non-specific substitution patterns are shown in Formulas 38-50 below, but it is to be appreciated that even more specific examples may feature R¹, Z and Q at any open valence position upon either of the fused phenyl rings.

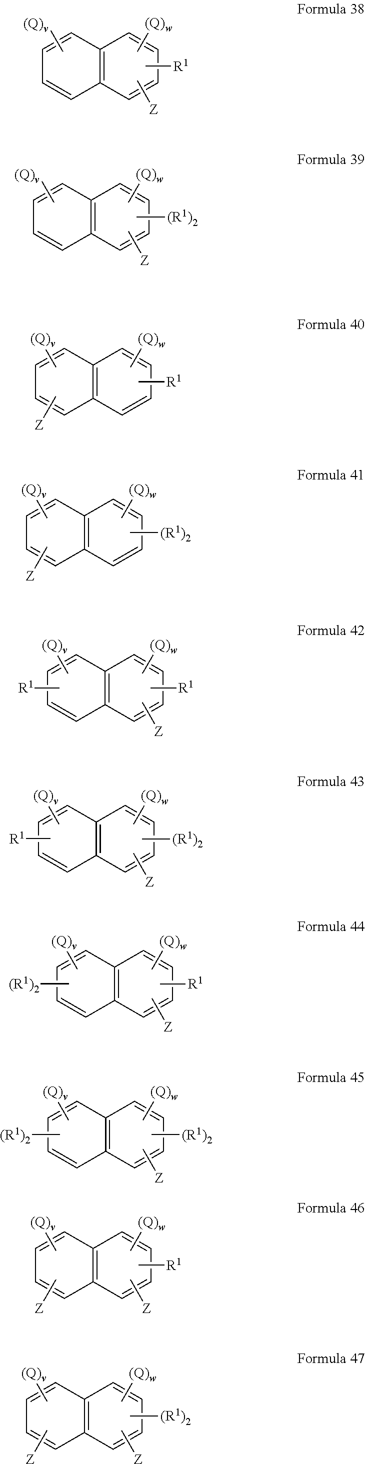

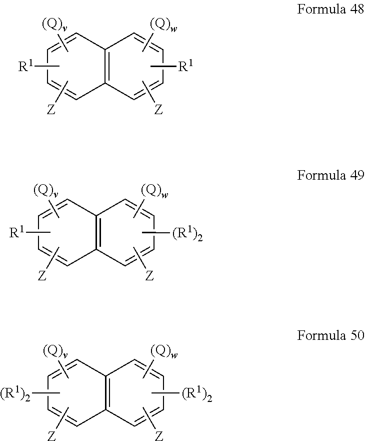

Particular embodiments of Formulas 38-50 include those in which at least one of variables v or w is zero. In some or other embodiments, variables v and w are both zero.

The surfactant compositions of the present disclosure may be formulated in solid form or dispersed in a suitable fluid phase. The fluid phase may comprise an aqueous fluid In any embodiment.

Accordingly, surfactant compositions of the present disclosure may further comprise an aqueous fluid in which the one or more amphiphilic compounds are dissolved. Suitable aqueous fluids are not particularly limited and may be selected from deionized water, tap water, fresh water, surface water, ground water, brackish water, salt water, sea water, brine, to or any combination thereof. Other aqueous fluid sources may also be suitable. The aqueous fluid may further comprise a water-miscible organic solvent such as alcohols, for example, In any embodiment.

When dissolved in a suitable aqueous fluid, the amphiphilic compounds disclosed herein may exhibit a range of surfactant properties. According to some embodiments, the amphiphilic compounds may be present in the aqueous fluid above a critical micelle concentration.

It is to be appreciated that numerous synthetic methods may be applied for synthesizing the amphiphilic compounds disclosed herein. Such methods may be envisioned by one having ordinary skill in the art of organic synthetic methodology. Illustrative methods of synthesizing the amphiphilic compounds are provided hereinafter.

For example, various biaryl compounds, such as those shown in Formulas 8-21, may be synthesized by palladium-mediated Suzuki coupling between an arylboronic acid and an aryl halide, particularly an aryl bromide or aryl iodide. The hydrophobic moieties (R¹), hydrophilic moieties (Z), and optional functional groups (Q) may be elaborated in the starting materials, or they may be introduced after the two phenyl rings have been coupled together. The related bis-furan compounds (Formulas 23-36) and bis-thiophenes may be synthesized through similar types of reactions.

Suzuki coupling of an alkenyl boronic acid and an aryl halide may similarly be used to couple cycloaliphatic rings to an aryl halide to form compounds having structures similar to those shown in Formula 22. Again, the hydrophobic and/or hydrophilic moieties may be introduced before or after coupling takes place. Reduction of the double bond in the cycloaliphatic ring (e.g., by hydrogenation) following Suzuki coupling may be used to form a saturated variant of the cycloaliphatic ring.

In any embodiment, Ullmann coupling of two aryl halide molecules in the presence of copper may be used to form compounds having two phenyl rings bonded to one another.

Other techniques for forming biaryl-type compounds may include dehydrogenation to of a cycloaliphatic-substituted phenyl ring (e.g., cyclohexylbenzene). Cycloaliphatic-substituted phenyl rings may be made by various techniques, such as via hydroalkylation of benzene or a substituted phenyl ring in the presence of a molecular sieve catalyst, AlC₃ and other Lewis acids, or various phosphoric acids. Cyclohexylbenzene and cyclopentylbenzene compounds, for example, may be formed directly by hydroalkylation. Dehydrogenation may then be performed to form biaryl compounds.

Described herein is:

A. Surfactant compositions comprising a bicyclic scaffold having a sigma bond. The surfactant compositions comprise: one or more amphiphilic compounds having a structure of

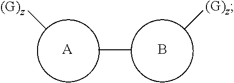

wherein A is an aromatic ring, a heteroaromatic ring, or a cycloaliphatic ring, each ring being defined by 5 to 10 ring atoms, B is an aromatic ring or a heteroaromatic ring, each ring being defined by 5 to 6 ring atoms, wherein at least one G is a hydrophobic moiety and at least one G is a hydrophilic moiety, and z is 0 or a positive integer ranging up to the number of ring atoms in each of A and B; and wherein the hydrophobic moiety comprises branched or unbranched C₆ to C₃₀ alkyl or alkenyl group, and the hydrophilic moiety comprises a polar functional group selected from the group consisting of quaternary ammonium, sulfonate, sulfate, carboxylate, phosphate, and ethoxylate.

B. Surfactant compositions comprising a naphthalene bicyclic scaffold. The compositions comprise: one or more amphiphilic compounds having a structure of

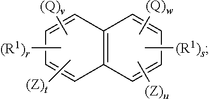

wherein R¹ is a branched or unbranched C₆ to C₃₀ alkyl or alkenyl group, such as a vinylidene olefin, each occurrence of R¹ being the same or different; wherein Z is selected from the group consisting of quaternary ammonium, sulfonate, sulfate, carboxylate, phosphate, and ethoxylate, each occurrence of Z being the same or different; wherein Q is selected from the group consisting of a C₁ to C₄ alkyl group, a C₁-C₄ alkoxy group, and a halide, each occurrence of Q being the same or different; wherein r and s are integers greater than or equal to zero, with the proviso that at least one of r or s is not zero; wherein t and u are integers greater than or equal to zero, with the proviso that at least one of t or u is not zero; and wherein v and w are to independently 0 or 1; wherein r+t+v is 4 or less and s+u+w is 4 or less.

Embodiments A and B may have one or more of the following additional elements in any combination:

Element 1: wherein the one or more amphiphilic compounds have a structure of

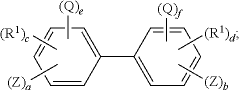

wherein R¹ is a branched or unbranched C₆ to C₃₀ alkyl or alkenyl group, each occurrence of R¹ being the same or different; wherein Z is selected from the group consisting of quaternary ammonium, sulfonate, sulfate, carboxylate, phosphate, and ethoxylate, each occurrence of Z being the same or different; wherein Q is selected from the group consisting of a C₁ to C₄ alkyl group, a C₁ to C₄ alkoxy group, and a halide, each occurrence of Q being the same or different; wherein a and b are integers greater than or equal to zero, with the proviso that at least one of a or b is not zero; wherein c and d are integers greater than or equal to zero, with the proviso that at least one of c or d is not zero; and wherein e and f are independently 0 or 1; wherein a+c+e is 5 or less and b+d+f is 5 or less.

Element 2: wherein the one or more amphiphilic compounds have a structure selected from the group consisting of:

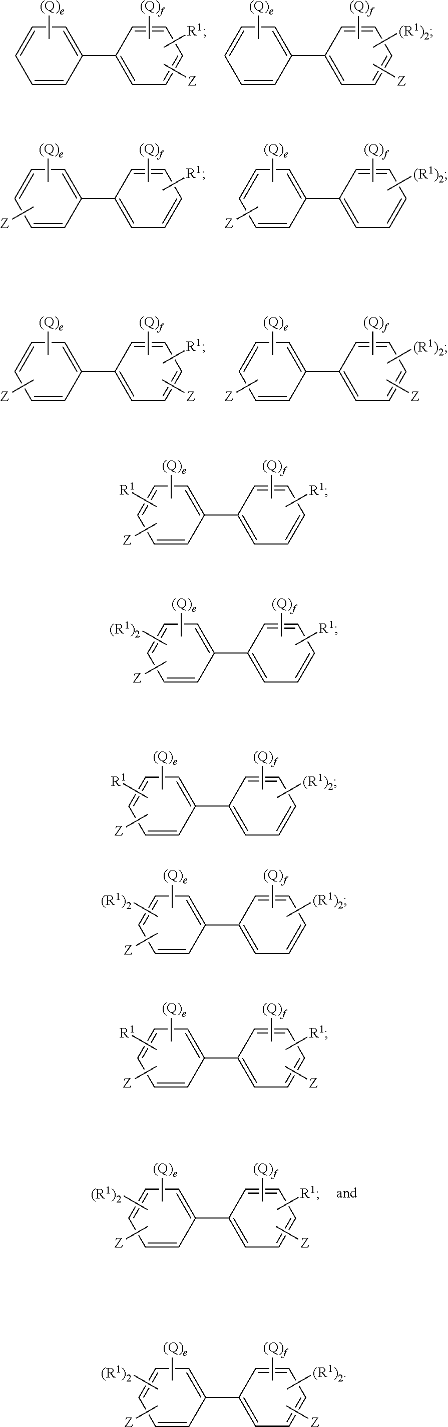

Element 3: wherein at least one of e or f is 0.

Element 4: wherein e and f are 0.

Element 5: wherein each occurrence of Z is sulfonate.

Element 6: wherein the one or more amphiphilic compounds have a structure of

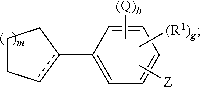

wherein R¹ is a branched or unbranched C₆ to C₃₀ alkyl or alkenyl group, such as a vinylidene olefin; wherein Z is selected from the group consisting of quaternary ammonium, sulfonate, sulfate, carboxylate, phosphate, and ethoxylate; wherein Q is selected from the group consisting of a C₁ to C₄ alkyl group, a C₁ to C₄ alkoxy group, and a halide; wherein g is 1 or 2; wherein h is 0 or 1; and wherein m is an integer ranging from 1 to 6, thereby forming a optionally unsaturated 5- to 10-membered cycloaliphatic ring selected from the group consisting of cyclopentyl, cyclohexyl, cycloheptyl, cyclooctyl, cyclononyl, cyclodecyl, cyclopenten-1-yl, cyclohexen-1-yl, cyclohepten-1-yl, cycloocten-1-yl, cyclononen-1-yl, and cyclodecen-1-yl.

Element 7: wherein the one or more amphiphilic compounds have a structure

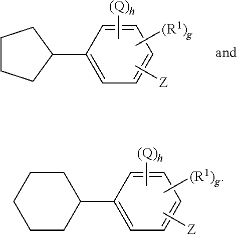

selected from the group consisting of

Element 8: wherein Z is sulfonate.

Element 9: wherein the one or more amphiphilic compounds have a structure of

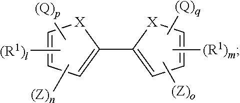

wherein R¹ is a branched or unbranched C₆ to C₃₀ alkyl or alkenyl group, each occurrence of R¹ being the same or different; wherein Z is selected from the group consisting of quaternary ammonium, sulfonate, sulfate, carboxylate, phosphate, and ethoxylate, each occurrence of Z being the same or different; wherein Q is selected from the group consisting of a C₁ to C₄ alkyl group, a C₁ to C₄ alkoxy group, and a halide, each occurrence of Q being the same or different; wherein X is O or S; wherein l and m are integers greater than or equal to zero, with the proviso that at least one of l and m is not zero; wherein n and o are integers greater than or equal to zero, with the proviso that at least one of n and or o is not zero; and wherein p and q are to independently 0 or 1; wherein n+l+p is 3 or less and o+m+q is 3 or less.

Element 10: wherein X is O.

Element 11: wherein the one or more amphiphilic compounds have a structure selected from the group consisting of:

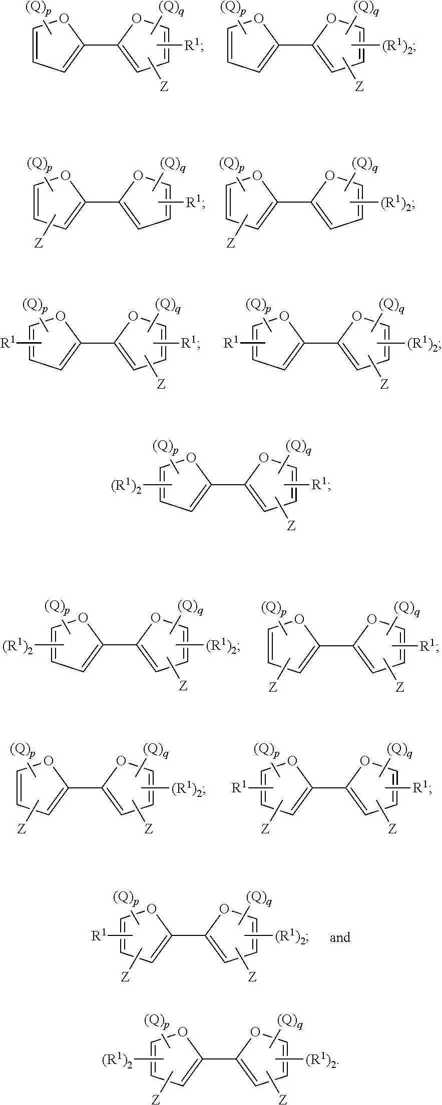

Element 12: wherein at least one of p and q is 0.

Element 13: wherein p and q are 0.

Element 14: wherein each occurrence of Z is sulfonate.

Element 15: wherein the surfactant composition further comprises an aqueous fluid in which the one or more amphiphilic compounds are dissolved.

Element 16: wherein the one or more amphiphilic compounds have a structure selected from the group consisting of:

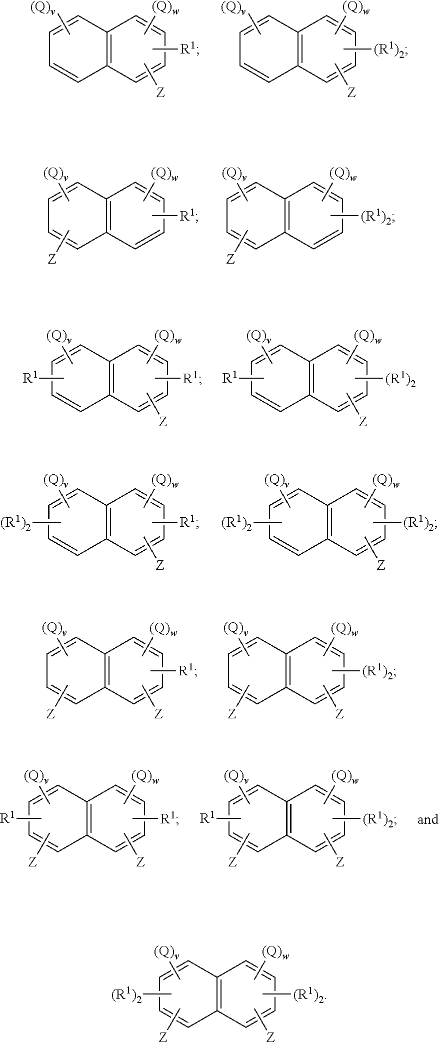

Element 17: wherein at least one of v and w is 0.

Element 18: wherein v and w are 0.

By way of non-limiting example, exemplary combinations applicable to A include: 1 and 2; 1 and 3; 1 and 4; 1 and 5; 1 and 15; 2 and 3; 2 and 4; 2 and 5; 2 and 15; 2, 3 and 5; 2, 4 and 5; 6 and 7; 6 and 8; 7 and 8; 6 and 15; 7 and 15; 8 and 15; 9 and 10; 9 and 11; 9 and 12; 9 and 13; 9 and 15; 9, 10 and 12; 9, 10, 12 and 15; 9, 10 and 13; 9, 10, 13 and 15; 9, 10 and 14; 9, 10, 14 and 15; 9, 11 and 12; 9, 11 and 13; 9, 11 and 14, 9, 11, 12 and 15; 9, 11, 13 and 15; and 9, 11, 14 and 15.

By way of non-limiting example, exemplary combinations applicable to B include: 14 and 15; 14 and 16; 14 and 17; 14 and 18; 15 and 16; 15 and 17; 15 and 18; 16 and 17; 16 and 18.

To facilitate a better understanding of the embodiments described herein, the following examples of various representative embodiments are given. In no way should the following examples be read to limit, or to define, the scope of the present disclosure.

### Examples

The performance properties of various surfactants having structures consistent with the disclosure herein were compared against sodium dodecylbenzenesulfonate (SDBS), a conventional linear alkylbenzene sulfonate surfactant. Formulas 51a, 51b, 52-55, 56a, and 56b show the structures of the compounds that were tested in the examples.

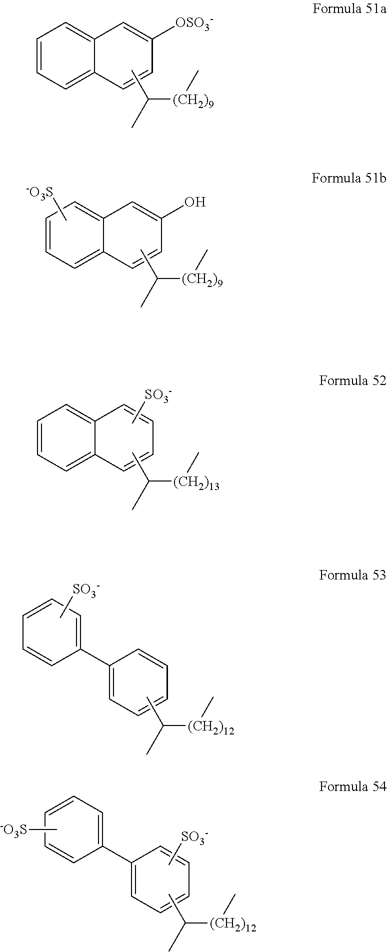

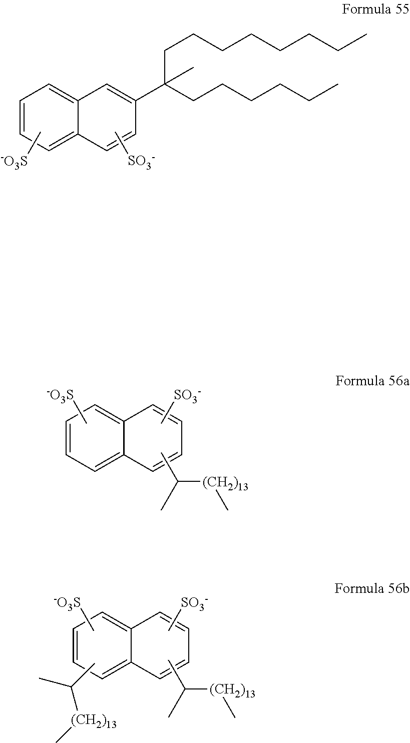

By way of non-limiting example, the compound of Formula 55 was prepared by alkylation of naphthalene with a C₁₆ vinylidene olefin having the structure of Formula 57, an internal vinylidene:

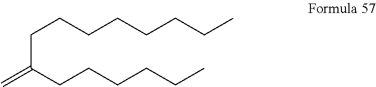 [SMILES: C=C(CCCCCC)CCCCCCCC]

In any embodiment, such internal vinylidenes, especially C₆ to C₂₀, or C₃₀ internal vinylidenes, are preferred. Alkylation was promoted by a zeolite catalyst under heating conditions in a Parr autoclave reactor. Product isolation was accomplished by filtration of the reaction mixture followed by vacuum distillation. Further details of the alkylation process may be found in Example 1 of US 2008/0234157. Thereafter, sulfonation was conducted by exposing the reaction product to SO₃ in a thin-film reactor. Characterization of the product as being disulfonated was based upon the quantity of SO₃ consumed during the sulfonation reaction.

Table 1 below summarizes the surfactant properties of sulfonate surfactants of the present disclosure (Formulas 51a/51b, 52-55, and 56a/56b) against those of sodium dodecylbenzenesulfonate.

TABLE 1

| | | Foaming of | | | | | |
|---|---|---|---|---|---|---|---|
| | | 0.1 Wt. % | | Wetting of | Calcium | | |
| | | surfactant | | 0.1 Wt. % | Tolerance | IFT | |
| | | Initial | 5 min. | surfactant | (mg Ca/g | DI H₂O | 1% NaCl |
| Entry | Formula(s) | (mm) | (mm) | (sec) | sample) | (wt. %) | (wt. %) |
| 1 | 51a and 51b | 82 | 60 | 35 | 116 | 26 | 0.195 |
| 2 | 52 | 110 | 80 | >300 | 640 | 25.6 | 0.224 |
| 3 | 53 | 86 | 60 | >300 | 335 | 24.9 | 0.51 |
| 4 | 54 | 115 | 100 | >300 | soluble | 25.8 | 0.82 |
| 5 | 55 | 110 | 100 | 270 | soluble | 26 | 0.46 |
| 6 | 56a and 56b | 134 | 34 | 26.5 | 160 | 1.93 | — |
| 7 | SDBS | 124 | 119 | 5.2 | 225 | 2-3 | — |

| | | | | C₂₀ | | | |
|---|---|---|---|---|---|---|---|
| | CMC | | ST | (pC₂₀) | | | Surface |
| | DI H₂O | DI H₂O | DI H₂O | DI H₂O | DI H₂O | CMC/ | Tension |
| Entry | (wt. %) | (mol/L) | (nM/m) | (wt. %) | (mol/L) | C₂₀ | Effectiveness |
| 1 | 0.10 | 2.58 × 10⁻³ | 35.0 | 4.0 × 10⁻⁴ | 1.03 × 10⁻⁵ | 250.0 | 36.75 |
| | | | | (3.40) | (4.99) | | |
| 2 | 0.010 | 2.20 × 10⁻⁴ | 33.9 | 6.0 × 10⁻⁴ | 1.32 × 10⁻⁵ | 16.67 | 37.81 |
| | | | | (3.22) | (4.88) | | |
| 3 | 0.080 | 1.77 × 10⁻³ | 44.0 | 0.015 | 3.32 × 10⁻⁴ | 5.33 | 27.70 |
| | | | | (1.82) | (3.48) | | |
| 4 | 0.010 | 1.8 × 10⁻⁴ | 42.6 | 6.0 × 10⁻⁴ | 1.08 × 10⁻⁵ | 16.67 | 29.15 |
| | | | | (3.22) | (4.97) | | |
| 5 | 0.010 | 2.18 × 10⁻⁴ | 34.2 | 1.8 × 10⁻³ | 3.92 × 10⁻⁵ | 5.56 | 37.48 |
| | | | | (2.74) | (4.41) | | |
| 6 | 9 × 10⁻⁴ | 1.60 × 10⁻⁵ | 33.0 | 1.5 × 10⁻⁵ | 2.60 × 10−⁷ | 60.0 | 38.70 |
| | | | | (4.82) | (6.59) | | |
| 7 | 0.01-0.05 | — | — | 0.012 | 3.45 × 10⁻⁴ | 2-3 | — |
| | | | | (1.92) | (3.46) | | |

Surfactant activities were measured using conditions specified in ASTM D3049. Critical micelle concentrations in water and 1% NaCl solution, surface tension values at the critical micelle concentration, and C₂₀ values were measured using conditions specified in ISO 4311. Foaming properties were measured using conditions specified in ASTM D1173-07. Wetting was measured using conditions specified in ASTM D2281. Interfacial tension (IFT) values were measured using conditions specified in ASTM D1331. Surface tension effectiveness represents the difference between the measured surface tension in water and the surface tension to of water itself (71.7 mN/m). C₂₀ values represent the surfactant concentration needed to decrease the surface tension of the solvent by 20 mN/m.

Calcium tolerance was tested using the following procedure. A 0.1 wt. % solution of the surfactant was prepared by dissolving 0.050 g of the surfactant in 50 mL of distilled water in a 200 mL Erlenmeyer flask. This solution was used as a blank to set a turbidity value of 0 on a LaMotte 2020 Turbidity Meter. A 1.00 wt. % solution of calcium chloride was titrated into the surfactant solution in 0.20 mL increments using a 5 mL micro-buret. The solution was then mixed and the turbidity was read again after each calcium chloride aliquot addition. The haze reading was then plotted against the titer (volume of added calcium chloride solution). The amount of added calcium chloride solution needed to produce a haze reading of 50 was then determined. This reading represents the lowest perceptible haze. The titer volume and concentration and the sample concentration may then be used to determine the number of milligrams of calcium that may be tolerated per gram of sample before haziness occurs.

As shown in Table 1, several of the surfactants (Entries 2-5) showed considerably higher calcium tolerance than did SDBS (Entry 7). Interfacial tension values for these surfactants were also higher than those of SDBS. Some of the surfactants remained soluble under the conditions for calcium exposure (Entries 4 and 5). C₂₀ values for the surfactants of Entries 1, 2 and 4-6 also occurred at lower concentrations than did those of SDBS, and the C₂₀ value for the surfactant of Entry 3 was comparable to that of SDBS.

As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Whenever a numerical range with a lower limit and an upper limit is disclosed, any number and any included range falling within the range is specifically disclosed. In particular, every range of values (of the form, “from a to b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1-16. (canceled)

17. A surfactant composition comprising:
one or more amphiphilic compounds having a structure of 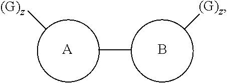
wherein A is an aromatic ring, a heteroaromatic ring, or a cycloaliphatic ring, each ring being defined by 5 to 10 ring atoms, B is an aromatic ring or a heteroaromatic ring, each ring being defined by 5 to 6 ring atoms, wherein at least one G is a hydrophobic moiety and at least one G is a hydrophilic moiety, and z is 0 or a positive integer ranging up to the number of ring atoms in each of A and B; and
wherein the hydrophobic moiety comprises branched or unbranched C₆ to C₃₀ alkyl or alkenyl group, and the hydrophilic moiety comprises a polar functional group selected from the group consisting of quaternary ammonium, sulfonate, sulfate, carboxylate, phosphate, and ethoxylate.

18. The surfactant composition of claim 17, wherein the one or more amphiphilic compounds have a structure of 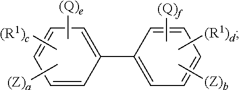
wherein R¹ is a branched or unbranched C₆ to C₃₀ alkyl or alkenyl group, each occurrence of R¹ being the same or different;
wherein Z is selected from the group consisting of quaternary ammonium, sulfonate, sulfate, carboxylate, phosphate, and ethoxylate, each occurrence of Z being the same or different;
wherein Q is selected from the group consisting of a C₁ to C₄ alkyl group, a C₁ to C₄ alkoxy group, and a halide, each occurrence of Q being the same or different; wherein a and b are integers greater than or equal to zero, with the proviso that at least one of a or b is not zero;
wherein c and d are integers greater than or equal to zero, with the proviso that at least one of c or d is not zero; and
wherein e and f are independently 0 or 1;
wherein a+c+e is 5 or less and b+d+f is 5 or less.

19. The surfactant composition of claim 18, wherein the one or more amphiphilic compounds have a structure selected from the group consisting of:

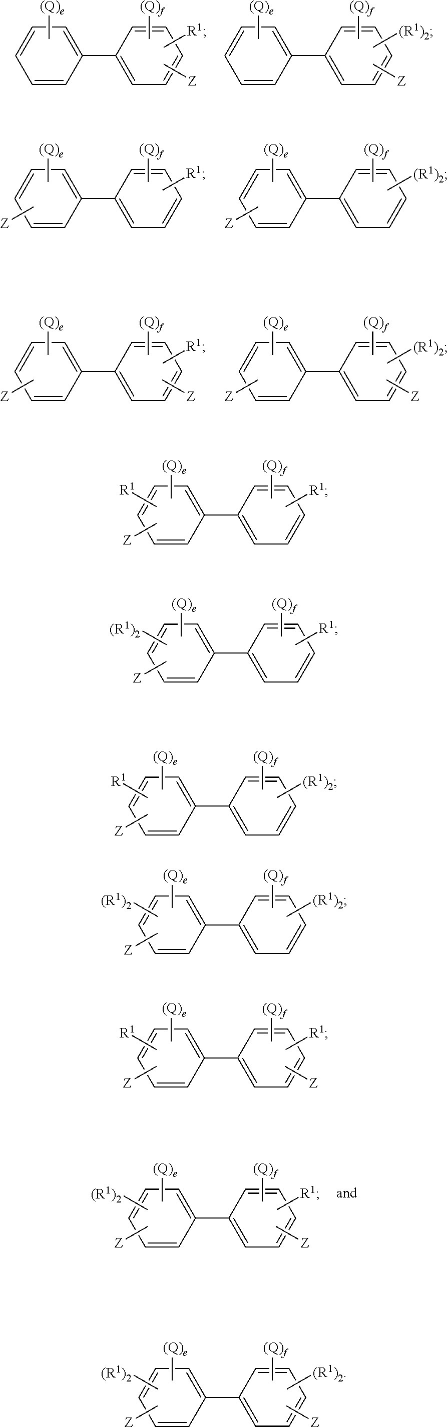

20. The surfactant composition of claim 18, wherein at least one of e or f is 0.

21. The surfactant composition of claim 18, wherein e and f are 0.

22. The surfactant composition of claim 18, wherein each occurrence of Z is sulfonate.

23. The surfactant composition of claim 17, wherein the one or more amphiphilic compounds have a structure of 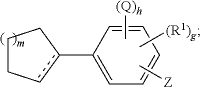
wherein R¹ is a branched or unbranched C₆ to C₃₀ alkyl or alkenyl group;
wherein Z is selected from the group consisting of quaternary ammonium, sulfonate, sulfate, carboxylate, phosphate, and ethoxylate;
wherein Q is selected from the group consisting of a C₁ to C₄ alkyl group, a C₁ to C₄ alkoxy group, and a halide;
wherein g is 1 or 2;
wherein h is 0 or 1; and
wherein m is an integer ranging from 1 to 6, thereby forming a optionally unsaturated 5- to 10-membered cycloaliphatic ring selected from the group consisting of cyclopentyl, cyclohexyl, cycloheptyl, cyclooctyl, cyclononyl, cyclodecyl, cyclopenten-1-yl, cyclohexen-1-yl, cyclohepten-1-yl, cycloocten-1-yl, cyclononen-1-yl, and cyclodecen-1-yl.

24. The surfactant composition of claim 23, wherein the one or more amphiphilic compounds have a structure selected from the group consisting of:

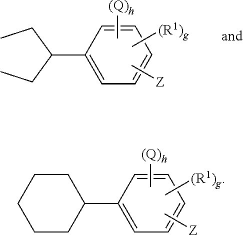

25. The surfactant composition of claim 23, wherein Z is sulfonate.

26. The surfactant composition of claim 17, wherein the one or more amphiphilic compounds have a structure of 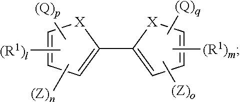
wherein R¹ is a branched or unbranched C₆ to C₃₀ alkyl or alkenyl group, each occurrence of R¹ being the same or different;
wherein Z is selected from the group consisting of quaternary ammonium, sulfonate, sulfate, carboxylate, phosphate, and ethoxylate, each occurrence of Z being the same or different;
wherein Q is selected from the group consisting of a C₁ to C₄ alkyl group, a C₁ to C₄ alkoxy group, and a halide, each occurrence of Q being the same or different; wherein X is O or S;
wherein l and m are integers greater than or equal to zero, with the proviso that at least one of l and m is not zero;
wherein n and o are integers greater than or equal to zero, with the proviso that at least one of n and or o is not zero; and
wherein p and q are independently 0 or 1;
wherein n+l+p is 3 or less and o+m+q is 3 or less.

27. The surfactant composition of claim 26, wherein X is O.

28. The surfactant composition of claim 26, wherein the one or more amphiphilic compounds have a structure selected from the group consisting of:

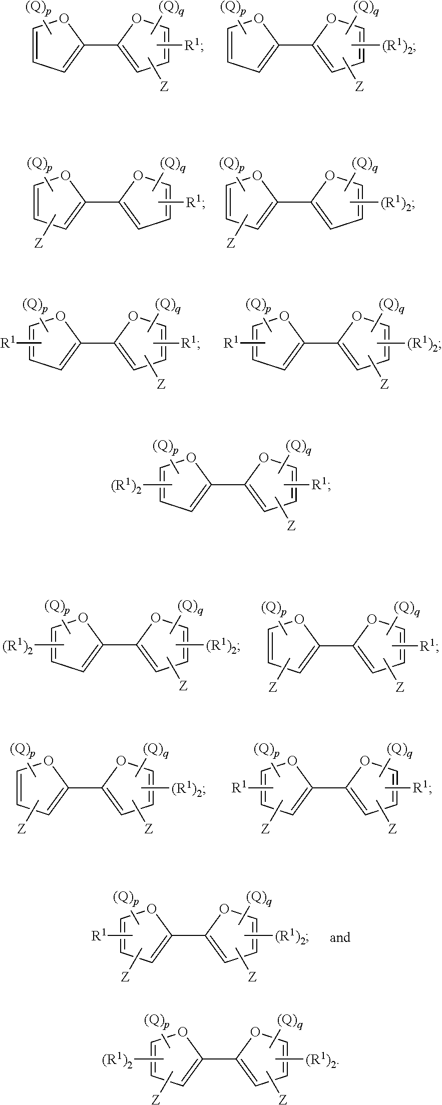

29. The surfactant composition of claim 26, wherein at least one of p and q is 0.

30. The surfactant composition of claim 26, wherein p and q are 0.

31. The surfactant composition of claim 26, wherein each occurrence of Z is sulfonate.

32. The surfactant composition of claim 17, further comprising an aqueous fluid in which the one or more amphiphilic compounds are dissolved.
